(19) United States
(12) Patent Application Publication
(10) Pub. No.: US 2005/0003008 A1
(43) Pub. Date: Jan. 6, 2005

(54) Method of in vivo drug targeting to solid tumors via acoustically triggered drug delivery in polymeric micelles

(76) Inventor: Natalya Rapoport, Sandy, UT (US)

Correspondence Address: ALAN J. HOWARTH, P.O. BOX 1909, SANDY, UT, 84091-1909, US

(21) Appl. No.: 10/722,379

(22) Filed: Nov. 24, 2003

Related U.S. Application Data
(63) Continuation-in-part of application No. 09/509,255, filed on Mar. 23, 2000, now abandoned.
(60) Provisional application No. 60/059,774, filed on Sep. 23, 1997.

(51) Int. Cl. A61K 9/14

(52) U.S. Cl. 424/486

(57) ABSTRACT

A method for administering a drug to a selected site in a patient includes (a) administering a composition including a micellar drug carrier having a hydrophobic core and an effective amount of the drug disposed in the hydrophobic core; and (b) applying ultrasonic energy to the selected site such that the drug is released from the hydrophobic core to the selected site. Illustrative drug carriers include ABA triblock copolymers, AB diblock copolymers, mixtures of ABA triblock copolymers and AB diblock copolymers, and mixtures of such polymers with PEGylated diacylphospholipids.

### CROSS-REFERENCE TO RELATED APPLICATIONS

This application is a continuation-in-part of U.S. patent application Ser. No. 09/509,255, filed Mar. 23, 2000, which is hereby incorporated in its entirety, which is the National Stage of International Application No. PCT/US98/20046, filed Sep. 23, 1998, which was published in English under PCT Article 21(2), which claimed the benefit of U.S. Provisional Application No. 60/059,774, filed Sep. 23, 1997.

### STATEMENT REGARDING FEDERALLY SPONSORED RESEARCH OR DEVELOPMENT

This invention was made with government support under Grant No. RO1 HL-52216 awarded by the National Institutes of Health. The government has certain rights in the invention.

### BACKGROUND OF THE INVENTION

This invention relates to drug delivery. More particularly, the invention relates to ultrasonically enhanced drug delivery using micellar drug carriers.

The efficacy of cancer chemotherapy is often limited by toxic side effects of the anticancer drugs. An ideal scenario would be to sequester the drug in a package that would have minimal interaction with healthy cells and would contain the drug until release. Then, at an appropriate time the drug would be released from the sequestering container at the tumor site. To achieve this goal, various long-circulating colloid drug delivery systems have been designed during the past three decades. A common structural motif of all these long-circulating systems, whether they be nanoparticles, liposomes, or micelles, is the presence of poly(ethylene oxide) (PEO) at their surfaces. The dynamic PEO chains prevent particle opsonization and render them “unrecognizable” by the reticuloendothelial system (RES) of cells. S. I. Jeon et al., 142 Colloid Interface Sci. 149-158 (1991). This advantage has promoted extensive research to develop new techniques to coat particles with PEO, techniques ranging from physical adsorption to chemical conjugation.

From a technological perspective, polymeric micelles formed by hydrophobic-hydrophilic block copolymers, with the hydrophilic blocks comprised of PEO chains, are very attractive drug carriers. These micelles have a spherical, core-shell structure with the hydrophobic block form the core of the micelle and the hydrophilic block or blocks forming the shell. Block copolymer micelles have promising properties as drug carriers in terms of their size and architecture. Only a few known block copolymers, however, form micelles in aqueous solutions. Among them, AB-type block copolymers (e.g. poly(L-amino acid)-co-poly(ethylene oxide) deserve special attention. M. Yokoyama et al., 51 Cancer Res. 3229-3236 (1991); G. Kwon et al., 9 Langmuir (1993); G. S. Kwon et al., 10 Pharma. Res. (970-974 (1993); G. S. Kwon et al., 6^{th} Int'l Symp. On Recent Advantages in Drug Delivery Systems 175-176 (1993); G. S. Kwon & K. Kataoka, 16 Adv. Drug Delivery Rev. 295-309 (1995)) and ABA-type triblock copolymers (e.g. A. V. Kabanov et al., 22 J. Controlled Rel. 141-158 (1992); V. Y. Alakhov et al., First Int'l Symp. On Polymer Therapeutics 213 (Univ. London 1996); A. V. Kabanov et al., 28 Macromolecules 2303-2314 (1995); 113 J. Magn. Res. A 65-73 (1995); N. Rapoport & K. Caldwell, 3 Colloids & Surfaces B: Biointerfaces 217-228 (1994); N. Rapoport, Eleventh Int'l Symp. On Surfactants in Solution 183 (Jerusalem 1996). The PLURONIC family of ABA-type triblock copolymers has the structure PEO-PPO-PEO, where PPO is poly(propylene oxide). The hydrophobic central PPO block forms a micelle core, and the flanking PEO blocks form the shell or corona that protects micelles from recognition by the RES.

Several advantages of polymeric micellar drug delivery systems include: (1) long circulation time in the blood and stability in biological fluids; (2) appropriate size (10-30 nm) to escape renal excretion but to allow for extravasation at the tumor site; (3) simplicity in drug incorporation compared to covalent bonding of the drug to a polymeric carrier; and (4) drug delivery independent of drug character.

Some micellar systems are dynamically stable because their solid-like cores dissociate slowly at concentrations below their critical micelle concentration (CMC). M. Yokoyama et al., 10 Pharma. Res. 895-899 (1993); K. Kataoka et al., 24 J. Controlled Rel. 119-132 (1993); A. Halperin & S. Alexander, 22 Macromolecules 2403-2412 (1989). Others are not stable and require additional stabilization that may be achieved, for instance by cross-linking the micelle core. A. Rolland et al., 44 J. Appl. Polym. Sci. 1195-1203 (1992); U.S. Pat. No. 6,649,702.

In a study of pharmacokinetics and distribution of doxorubicin in micelles formed by drug-polymer conjugates, the conjugates circulated in the form of micelles much longer in blood than did free drug. M. Yokoyama, 17^{th} Int'l Symp. On Recent Advantages in Drug Delivery Systems 99-102 (1995). The uptake of the conjugated drug by various organs proceeded much slower than that of a free drug, and lower levels of conjugate were found in the heart, lung, and liver compared to much higher conjugate level in the tumor. M. Yokoyama, Advances in Polymeric Systems for Drug Delivery (1994).

Cross-resistance to anti-cancer drugs in malignant cells is also a major problem for chemotherapy. M. S. Sanford & S. Melvin, 91 Proc. Nat'l Acad. Sci. USA 3497 (1994). Despite an initial favorable response to chemotherapy, almost 50% of patients relapse, and the recurrence of the disease is often associated with clinical drug resistance. P. Maslak et al., 17 Cytometry 84 (1994). The most common resistance mechanism is increased drug efflux due to amplification of the gene for P-glycoprotein. R. L. Juliano & V. Ling, 445 Biochim. Biophys. Acta 152 (1976); J. L. Biedler & H. Riehm, 30 Cancer Res. 1174 (1970); G. Bradley et al., 948 Biochim. Biophys. Acta 87 (1988). P-glycoprotein (P-gp) is situated in plasma membrane and acts as an energy-dependent drug-efflux pump producing decreased drug accumulation within the cells.

Several attempts have been made to overcome resistance in cancer cells. Drugs such as verapamil have been shown to modulate P-gp activity by inhibiting the binding of some anti-neoplastic drugs to P-gp. T. Tsuruo et al., 42 Cancer Res. 4730 (1982). Although a number of other agents have been shown to reverse the multiple drug resistance (MDR) phenotype, J. A. Moscow et al., Multi Drug Resistance, in Cancer Chemotherapy and Biological Response Modifiers 91 (H. M. Pinedo et al. eds. 1992), their clinical applicability toward resistant tumors has been restricted due to their toxicities. U. Consoli et al., 88 Blood 633-644 (1996).

Several other methods have been proposed to overcome drug resistance, based on bypassing the P-gp pump such as drug delivery in liposomes, combined delivery of drugs and surfactants, delivery in micelles, and delivery of polymer-drug conjugates.

Ultrasound has been used extensively for medical diagnostics and physical therapy. An advantage of ultrasound lies in the fact that it is non-invasive, and the energy can be controlled and focused easily, with the capability to penetrate deep into the tissue. Several reports have demonstrated enhanced cytotoxic response when ultrasound and chemotherapeutic agents were combined. R. Jeffers, 98 J. Acoust. Soc. Am. 2380 (1995); V. Mislik et al., 25 Free Radical Res. 13-22 (1996); V. Mislik et al., 20 Free Radical Biology and Medicine 129-138 (1996). The most prominent manifestation of this drug-ultrasound synergy was an increased drug uptake. There are also several hypotheses reported in the literature regarding the mechanism of ultrasonic enhanced activity of anthracycline drugs. A. H. Saad & G. M. Hahn, Heat Transfer in Bioengineering and Medicine (J. C. Chato et al. eds. 1987); A. H. Saad & G. M. Hahn, 49 Cancer Res. 5931-5934 (1989); A. J. Saad & G. M. Hahn, 18 Ultrasound Med. Biol. 715-723 (1992); R. J. Jeffers, Activation of Anti-cancer Drugs with Ultrasound, Ph.D. Dissertation, Univ. of Michigan (1995); P. Loverock et al., 63 Br. J. Radiol. 542-546 (1990); D. B. Tata et al., 3 Ultrasonics Sonochemistry 39-45 (1996). These reports are mainly concerned with acoustic-induced hypersensitization of drug-sensitive lines.

To suppress side effects to normal tissue and to improve the efficiency towards the cancerous cells, targeting of these drugs using several types of drug carriers has been studied. The recent efforts towards designing such types of delivery systems have led to the development of delivery vehicles that are more stable in the blood system compared to previous carriers that were rapidly taken up by the reticuloendothelial system. Poly(ethylene oxide) (PEO) is a common structural component of these new drug carriers. It is a well known biomedical polymer, expresses low toxicity, and when present at surfaces and interfaces, it has the ability to suppress cellular and protein adsorption. G. S. Kwon et al., 2 Colloids Surfaces B: Biointerfaces 429-434 (1994). Poly(oxyethylene-b-oxypropylene-b-oxyethylene) triblock copolymers represent non-toxic polymeric surfactants that have been used in a number of drug targeting applications. V. Y. Alakhov et al., 7 Bioconjugate Chem. 209-216 (1996); A. V. Kabanov et al., 22 J. Controlled Rel. 141-158 (1994). These triblock polymers attract special attention due to their low toxicity and ability to solubilize biologically active lipophilic substances. I. R. Shmolka, 54 J. Am. Oil Chem. Soc. 110-116 (1977); E. W. Merril, Poly(ethylene oxide) and Blood Contact, in Poly(Ethylene Glycol) Chemistry 199-220 (J. M. Harris ed 1992). The concept underlying these polymers is the principle that the structure formed with amphipathic molecules will, in aqueous medium, present their hydrophilic (PEO) portion to the external aqueous media, while the hydrophobic parts (polypropylene oxide; hereinafter “PPO”) will be oriented towards the internal part of the structure. The hydrophobic drug molecules would then partition inside the micelles. It has been suggested that these 15-35 nm diameter carriers can enter the cells by phagocytosis or endocytosis, and the drug can be delivered inside the cells by local delivery or by fusion with the membrane, thereby destabilizing it. R. Paradis et al., 5 Int. J. Oncol. 1305-1308 (1994).

The structural transitions of one such triblock copolymer (PLURONIC P-105) has been reported. N. Rapoport & K. Caldwell, 3 Colloids and Surfaces B: Biointerfaces 217-228 (1994). The transition was shown to proceed from unimers to loose hydrated aggregates to stable dense micelles with a hydrophobic core. The onset of multimolecular micelles was shown to correspond to a concentration of 1 wt % of PLURONIC P-105 and was completed at 10 wt %, with two populations of micelles co-existing at intermediate concentrations. The solubilization efficiency of PLURONIC for hydrophobic or amphiphilic molecules was found to increase dramatically upon formation of dense micelles.

In view of the foregoing, it will be appreciated that providing a method for delivering drugs that avoids or reduces the side effects and multiple drug resistance phenomenon associated with many chemotherapeutic agents would be a significant advancement in the art.

### BRIEF SUMMARY OF THE INVENTION

It is a feature of the present invention to provide a method for delivering chemotherapeutic agents that avoids or reduces side effects and multiple drug resistance associated therewith.

It is also a feature of the invention to provide a drug delivery composition for treating cancer.

It is another feature of the invention to provide a method for delivering hydrophobic therapeutic agents by encapsulation in micelles in conjunction with ultrasound.

These and other features can be addressed by providing a method for delivery of a drug to a selected site in a patient comprising:
(a) administering to the patient a composition comprising a micellar drug carrier having a hydrophobic core and an effective amount of the drug disposed in the hydrophobic core; and
(b) applying ultrasonic energy to the selected site such that the drug is released from the hydrophobic core to the selected site.

A composition for delivery of a drug to a selected site in a patient comprises a micellar drug carrier having a hydrophobic core and an effective amount of the drug disposed in the hydrophobic core. Any polymeric micelles that are stable and retain their drug load in circulation can be used in the present invention. The micelles maybe formed of triblock copolymers, diblock copolymers, mixtures of triblock and diblock copolymers, or mixtures of such block copolymers with PEGylated diacylphospholipids. Illustratively, the micellar drug carriers are formed of mixed micelles of PEO-PPO-PEO triblock copolymers with 1,2-diacyl-sn-glycero-3-phosphoethanolamine-N-[methoxy(polyethylene glycol)] (“PEG-PE”), wherein the molecular weight of the poly(ethylene glycol) moiety is about 2000 to about 3000. Illustratively, the drug can be a hydrophobic drug or a drug having a hydrophobic center such that the drug can be sequestered in the hydrophobic core of the micellar carrier. Illustrative drugs include doxorubicin and ruboxyl.

A method of enhancing uptake of a drug by cells at a selected site in a patient comprises:
(a) administering to the patient a composition comprising a micellar drug carrier having a hydrophobic core and an effective amount of the drug disposed in the hydrophobic core; and
(b) applying ultrasonic energy to the selected site such that the drug is released from the hydrophobic core and taken up by the cells.

A method for reducing side effects in a patient from administration of a drug comprises:
(a) administering to the patient a composition comprising a micellar drug carrier having a hydrophobic core and an effective amount of the drug disposed in the hydrophobic core; and
(b) applying ultrasonic energy to the patient such that the drug is released from the hydrophobic core.

### BRIEF DESCRIPTION OF THE SEVERAL VIEWS OF THE DRAWINGS

FIG. 1 shows the concentration of solubilized DSTA as a function of PLURONIC P-105 concentration; EPR spectra before and after dense micelle formation are shown in the left inset and right insets, respectively.

FIG. 2 shows the structure of doxorubicin (DOX) and ruboxyl (Rb).

FIG. 3 shows the effect of temperature and PLURONIC P-105 concentration on ruboxyl fluorescence intensity (ruboxyl concentration=10 μg/ml): shaded, 25° C.; hatched, 37° C.; stippled, 42° C.

FIG. 4 shows the effect of ruboxyl encapsulation in PLURONIC P-105 on the drug uptake by HL-60 cells: fluorescence of HL-60 cell lysate normalized to the cell concentration as a function of PLURONIC P-105 concentration; ruboxyl concentration=40 μg/ml, 1 hour.

FIG. 5 shows that doxorubicin encapsulation in PLURONIC micelles restricts drug intercalation into DNA: fraction of retained fluorescence on doxorubicin intercalation into DNA as a function of PLURONIC P-105 concentration; doxorubicin concentration=10 μg/ml, DNA concentration=11 μg/ml.

FIG. 6 shows drug release from micelles on application of low frequency and high frequency ultrasound.

FIG. 7 shows doxorubicin fluorescence in the lysates of HL-60 cells incubated without sonication (shaded) or sonicated (hatched) with doxorubicin (20 μg/ml) for 1 hour, normalized to the cell concentration.

FIG. 8 shows uptake of DOX by ovarian carcinoma drug-sensitive A2780 cells after treatment with ultrasound.

FIG. 9 shows flow cytometry histograms of MDR A2780/ADR cells unsonicated or sonicated in the presence of PLURONIC micelles.

FIG. 10 shows growth inhibition of MDR cells after treatment with DOX and ultrasound.

FIG. 11 shows a fluorescence histogram of sonicated and unsonicated A2780 tumor cells treated in vivo with DOX.

FIG. 12 shows efficiency of ultrasound-enhanced drug delivery with PLURONIC P-105, PEG-PE, and a mixture of PLURONIC P-105 and PEG-PE.

FIG. 13 shows the survival rates of ovarian carcinoma-bearing mice for untreated control (▴), conventional treatment by 3 mg/kg doxorubicin (DOX) administered intraperitoneally in physiological solution (▪), and treatment by 3 mg/kg DOX delivered intraperitoneally in PLURONIC micelles in combination with ultrasound (⋄); ultrasound (1 MHz, 30 s, 1.2 W/cm²) was applied one hour after injection of drug.

### DETAILED DESCRIPTION

Before the present compositions and methods for drug delivery are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to a composition containing “a drug” includes a mixture of two or more drugs, reference to “a copolymer” includes reference to one or more of such copolymers, and reference to “a micelle” includes reference to a mixture of two or more micelles.

In describing and claiming the present invention, the following terminology will be used in accordance with the definitions set out below.

As used herein, “effective amount” means an amount of a drug or pharmacologically active agent that is nontoxic but sufficient to provide the desired local or systemic effect and performance at a reasonable benefit/risk ratio attending any medical treatment.

As used herein, “administering” and similar terms mean delivering the composition to the individual being treated such that the composition is capable of being circulated systemically to the parts of the body where the drug is to act, such as the site of the tumor. Thus, the composition is preferably administered to the individual by systemic administration, typically by subcutaneous, intramuscular, or intravenous administration, or intraperitoneal administration. Injectables for such use can be prepared in conventional forms, either as a liquid solution or suspension or in a solid form suitable for preparation as a solution or suspension in a liquid prior to injection, or as an emulsion. Suitable excipients include, for example, water, saline, dextrose, glycerol, ethanol, and the like; and if desired, minor amounts of auxiliary substances such as wetting or emulsifying agents, buffers, and the like can be added.

The most common and accepted form of cancer treatment, chemotherapy, is often limited by its deleterious side effects on normal tissues and a host of other problems, all of which compromise the patient's health. Therefore, a desirable improvement would be to reduce the dosage or frequency of drug administration by improving the effectiveness of drugs at the targeted site. It is shown herein that the combination of ultrasound and micellar drug carriers can lower the effective dosage of an anti-cancer drug, which provides a way to reduce the toxic side effects associated with high doses of chemotherapeutic drugs. The interaction of anti-cancer drugs with normal tissues can be circumvented by encapsulating the drug in polymeric micelles. Illustratively, PLURONIC P-105 is a non-toxic copolymer at concentrations much higher than the CMC and is not recognized by RES, although its cytotoxicity on normal cells has yet to be determined. The use of ultrasound is advantageous in the sense that ultrasound is a non-invasive technique. Ultrasound can be focused at selected depths in soft tissue throughout the body. This approach is capable of depositing large amounts of ultrasonic energy into deep tumors. By taking advantage of the non-invasive technique of ultrasound and creating non-toxic micellar drug carriers, a new approach to drug targeting is provided.

In the drug delivery modality described herein, effective intracellular drug uptake at the tumor site is activated by focused ultrasound. Ultrasonic waves can be directed to and focused on a particular volume of tissue. The depth of penetration and the shape of the energy deposition pattern may be controlled by varying the ultrasound frequency and the type and shape of the transducer. Optimal power densities at the target site may be obtained by adjusting the output power of the ultrasonic transducer.

It was found that ultrasound induced partial drug release from micelles and enhanced the intracellular uptake of both free and micellar-encapsulated drugs.

Both low-frequency and high-frequency ultrasound proved effective in triggering drug release from micelles (Example 4, FIG. 6). Low frequency ultrasound was found more effective in triggering drug release from micelles but it does not allow sharp focusing. In contrast, high frequency ultrasound allows sharp focusing but does not penetrate as deep in the interior of the body. A higher frequency ultrasound allows sharper focusing; however, drug release from micelles proceeds more effectively at lower frequencies. Therefore, optimal design of ultrasound treatment should depend on the tumor size and location. For instance, for tumors 2 cm in diameter or larger, application of 100 kHz (or a lower frequency) ultrasound appears quite feasible; optimal power densities could be achieved by controlling output energy. For smaller tumors that are not deep, a higher frequency ultrasound may be used since it provides for sharper focusing; power densities produced at the focal site by existing hyperthermia devices appear sufficient to cause drug release from micelles.

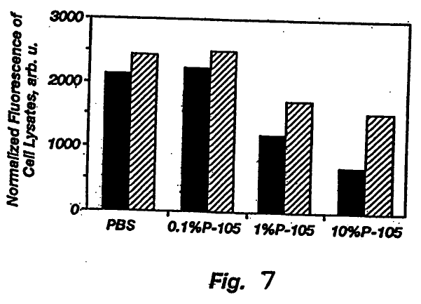

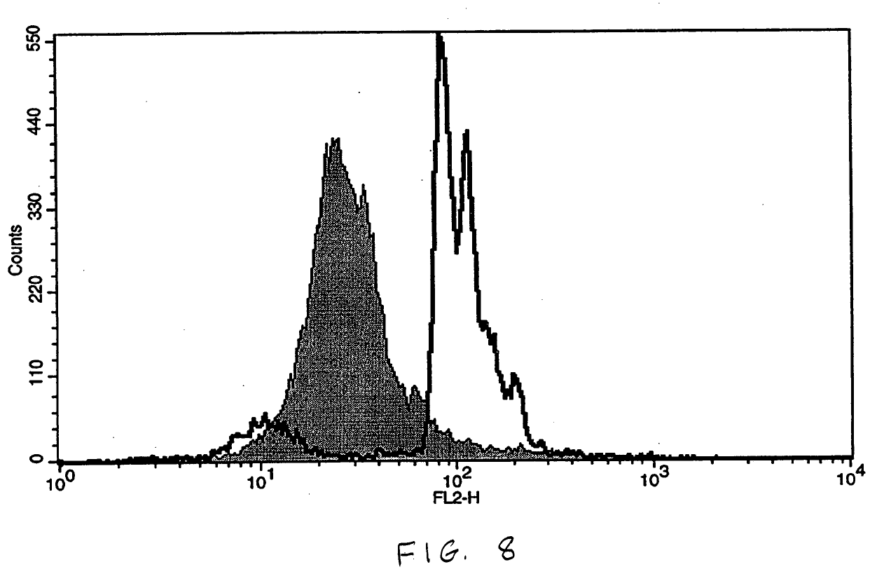

Both low frequency and high frequency ultrasound enhanced the intracellular drug uptake (Examples 5, 6, and 7; FIGS. 7, 8, and 9)).

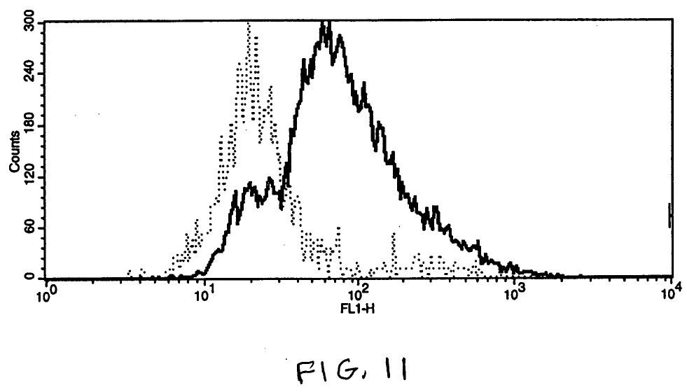

The combined micellar drug delivery and sonication resulted in a significant sensitization of the multidrug resistant (MDR) cells to the action of drugs (Example 8, FIG. 10).

The combined micellar drug delivery and sonication resulted in a significant sensitization of the multidrug resistant (MDR) cells to the action of drugs (Example 8, FIG. 10). In the MDR cells, drug encapsulation in PLURONIC micelles noticeably enhanced the inhibition of cell growth (FIG. 10). The effect was partly due to the cytostatic action of PLURONIC micelles; however, the cytotoxic action of the drug in the presence of PLURONIC micelles was also clearly enhanced.

It should be noted that because of the cytostatic action of micelles, the inhibitory concentration IC₅₀ values for drugs become meaningless; for example, upon 3-hour incubation with a drug-free 10% P-105 micelles followed by three days culturing in a PLURONIC-free RPMI 1640 medium, the growth inhibition of both sensitive and resistant cells exceeded 50%. Introduction of drug additionally inhibited cell growth. For cases like this it would be more informative to compare, e.g., IC₇₅ values. The corresponding data are presented in Table 1.

This comparison showed that without PLURONIC micelles, IC₇₅ of the drug (either Rb or DOX) in the MDR ovarian carcinoma cells was not achieved even at drug concentrations as high as 100 μg/ml; in the presence of 10% PLURONIC micelles, IC₇₅ of drugs in the MDR cells dropped to 16-17 μg/ml, but remained about an order of magnitude higher than in drug-sensitive ovarian carcinoma cells.

TABLE 1
IC₇₅ values for DOX and Rb in the drug-sensitive and MDR
ovarian carcinoma cells in the presence and absence of Pluronic
unimers and micelles.

| | DOX, μg/ml | | | Rb, μg/ml | | |
|---|---|---|---|---|---|---|
| Cell | | P-105 | P-105 | | P-105 | P-105 |
| Phenotype | RPMI | 0.1% | 10% | RPMI | 0.1% | 10% |
| A2780 | 1.3 | 1.3 | 1.3 | 1.5 | — | 0.9 |
| | ±0.4 | ±0.4 | ±0.4 | ±0.5 | | ±.5 |
| A2780/ADR | >100 | 12 ± 2 | 16 ± 2 | >100 | — | 17 ± 3 |

Growth inhibition of the MDR cells presumably resulted from the cytostatic effect of Pluronic micelles combined with the cytotoxic effect of drug; in many cases, these two factors acted synergistically.

Effect of Ultrasound on the Growth Inhibition of MDR Cells.

Ultrasonic irradiation of both drug-sensitive and MDR cells substantially reduced cell viability; this was true for free, unimer-associated, micellar-encapsulated drugs and for drug-free micelles. As an example, when the MDR cell were incubated with drug-free 10% PLURONIC micelles for three hours, upon which they were sonicated for 10 minutes by 69-kHz ultrasound at 3.2 W/cm² and cultured for 72 hours, the growth of the MDR cell dropped to forty percent of the unsonicated non-incubated control.

For the MDR cells, PLURONIC and ultrasound effects are especially important. At the absence of micelles, only about forty percent of the MDR cells are killed at DOX or Rb concentrations as high as 100 μg/ml. However, when the MDR cells were incubated with 5 μg/ml DOX for three hours in the presence of a unimeric Pluronic solution followed by a 10-minute sonication by 69-kHz ultrasound at 3.2 W/cm², sixty six percent of the cells died upon subsequent cell culturing vs. fifty three percent without ultrasound; only fifteen percent of the MDR cells die upon incubation with 5 μg/ml DOX without Pluronic and ultrasound treatment.

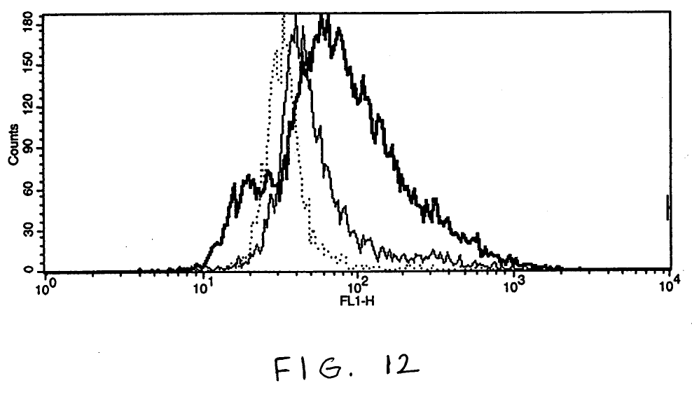

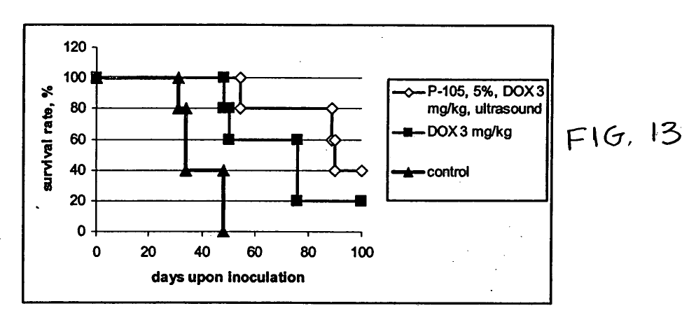

In vivo ultrasound induced a dramatic enhancement of the intracellular drug uptake by ovarian carcinoma tumor cells from polymeric micelles thus confirming tumor targeting (Examples 9 and 10, FIGS. 11, 12).

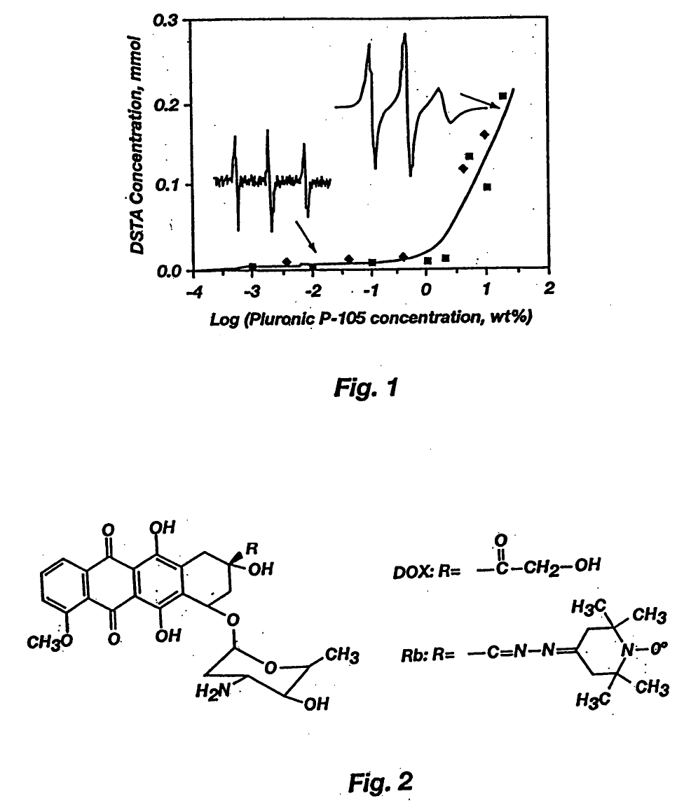

The EPR technique has been used previously to screen various members of the PLURONIC family of triblock copolymers to determine their micellization behavior. N. Rapoport & K. Caldwell, 3 Colloids and Surfaces B: Biointerfaces 217-228 (1994). A lipophilic spin probe, 16-doxyl stearic acid (DSTA or 16-DS) was used to report the hydrophobicity of the micelle core and the solubilization efficiency of PLURONIC micelles. PLURONIC P-105 was found, depending on the concentration, to exhibit three regions on a phase diagram corresponding to unimers, loose aggregates, and dense micelles. At the onset of dense multimolecular micelle formation, the PLURONIC solubilization efficiency for lipophilic substances increased dramatically (FIG. 1).

The EPR technique was used to determine the characteristics of various copolymers. Based on the EPR study, mixed micelles formed from a 5% solution of PLURONIC P-105 triblock copolymer mixed with a 5% solution of PEG-PE were chosen as an illustrative micellar system according to the present invention. These mixed micelles solubilized 50% more drug than pure PLURONIC micelles of the same PLURONIC concentration. They retained more than half of their drug load upon 50-fold dilution with blood plasma. Further, molecular motion in the core of such mixed micelles was more active, which makes them more susceptible to the action of ultrasound.

Two anti-cancer drugs are used in the presently described experiments (FIG. 2). Doxorubicin (DOX; also known as adriamycin) is widely used in clinical practice as a chemotherapeutic agent. It is an intercalating drug that stacks between paired bases in DNA. A strong drug-DNA interaction is critical for the drug's cytotoxic effect. Like other anti-cancer drugs of the anthracycline family, however, doxorubicin is cardiotoxic due to the induced production of active oxygen radicals. W. B. Pratt et al., in The Anticancer Drugs 155-182 (Oxford Univ. Press 1994); N. M. Emanuel et al., 53 Russian Chem. Rev. 1121-1138 (1984); J. H. Doroshow, Role of Reactive Oxygen Production in Doxorubicin in Cardiac Toxicity (Martinus Nijhoff Pub. 1988).

A paramagnetic analog of doxorubicin, i.e. ruboxyl (Rb), has a paramagnetic Tempo-type nitroxide radical (1-oxo-2,2,6,6-piperidone-4-hydrazone) conjugated to doxorubicin (FIG. 2). The nitroxide moiety in position 14 serves as a radical trap. Ruboxyl is both fluorescent and paramagnetic, which provides for fluorescence and EPR spectroscopy to be used independently of drug uptake, distribution, and metabolism. This makes ruboxyl a powerful research tool. The anti-tumor activity of ruboxyl on models of leukosis, La, P-388, and L-1210, inoculated on mice and on solid tumors in rats has been reported to be high. U.S. Pat. No. 4,332,934. In clinical trials the drug was found effective against breast and colon carcinomas and bone sarcoma, and cardiotoxicity was reduced.

### EXAMPLE 1

Micellization Using Ruboxyl and Doxorubicin as Fluorescent Probes

The anthraquinone moieties of ruboxyl and doxorubicin are inherently fluorescent, which makes it possible to use them as fluorescent probes. The fluorescence of both roboxyl and doxorubicin is quenched by collisions with water molecules. When ruboxyl and doxorubicin are prevented from colliding with water, their fluorescence increases about 3-fold. For example, at ruboxyl concentrations of 20 μg/ml, fluorescence intensity is 8200 (in arbitary units) in PBS and 29,800 in ethanol. This phenomenon was used to study the micellization process of various members of the PLURONIC family.

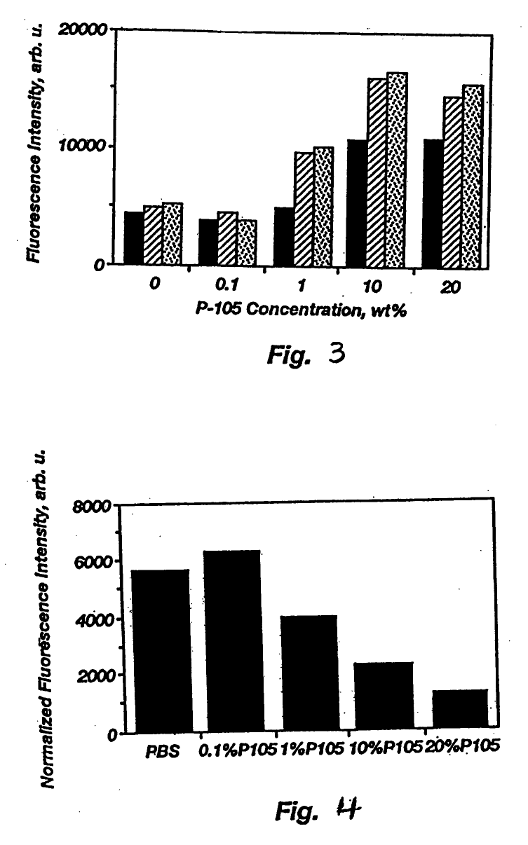

Technical fluorescence emission spectra were recorded over a temperature range of 25-42° C. using a photon counting spectrofluorometer (ISS, model PC-1, Champaign, Ill.). As could be expected, the ruboxyl fluorescence increased dramatically upon the onset of dense micelle formation in PLURONIC P-105 solutions (FIG. 3). Copolymer concentrations corresponding to the onset of dense micelle formation decreased with increasing temperature.

The solubilization efficiency of PLURONIC micelles for lipophilic compounds was monitored by the quantitative EPR technique using DSTA as a spin probe. PLURONIC solutions of various concentrations were incubated with DSTA powder at room temperature for 15 minutes under constant shaking. The non-solubilized fraction of the probe was separated by centrifugation. EPR spectra were collected from supernatants. The intensities (double integrals) of EPR spectra were compared to those of standard solutions.

The EPR spectra were recorded at room temperature with an X-band Bruker ER-200 SRC EPR spectrometer. Incident microwave power was set to 0.5-2 mW to avoid saturation. A modulation frequency of 100 kHz was used, and the modulation amplitude was typically a quarter of a linewidth.

EPR and fluorescence data were in good agreement in terms of copolymer concentration corresponding to the onset of dense micelle formation.

The additivity model may be used to analyze fluorescence intensity data:
Iₑₓₚ=aₘfₘ+aₛ(1−fₘ)
where aₘ and aₛ are quantum yields of probe fluorescence in hydrophobic and hydrophilic environments, respectively, and fₘ is the fraction of the probe located in the hydrophobic environment, i.e. in the hydrophobic micelle core. Free drug in solution and drug molecules associated with loose, water-penetrated PLURONIC P-105 aggregates are located in a hydrophilic environment.

Based on this model, the present data indicated that at 37° C. (the temperature of drug incubation with living cells) and in 1 wt % PLURONIC P-105 solutions, about 45% of the drug was localized in the hydrophobic environment, and in 10 wt % PLURONIC P-105 solutions 100% of the drug was localized in the hydrophobic environment.

### EXAMPLE 2

Drug Loading and Release from PLURONIC Micelles

To study solute release from PLURONIC micelles, the partitioning of the solute between the micelles and the surface of polystyrene latex particles was investigated. A spin probe, DSTA, or the drug, ruboxyl, was solubilized in PLURONIC P-105 solutions of various concentrations. A suspension of polystyrene latex particles (average diameter 0.9 μm, 50 μl/ml) was incubated with 1 ml of DSTA or ruboxyl solution in PLURONIC P-105, and depletion of the probe in the supernatant was measured by the EPR (for DSTA) or fluorescence (for ruboxyl) technique upon polystyrene particle separation.

Upon introduction into micellar PLURONIC solutions, doxorubicin and ruboxyl were spontaneously transferred into the inner core of the PLURONIC micelles. Free drug (if any) was removed by dialysis.

An important question pertinent to this research was how tightly the solubilized drug was associated with PLURONIC micelles. To investigate this problem, ruboxyl adsorption on polystyrene latex particles was measured from molecular solutions of ruboxyl in PBS and from micellar PLURONIC solutions. Ruboxyl readily adsorbs onto polystyrene surfaces. About 90% of the drug is transferred onto polystyrene surface from ruboxyl solutions in PBS. PLURONIC micelles, however, compete for ruboxyl with polystyrene surfaces; only about 40% of ruboxyl solubilized in PLURONIC micelles (20 wt % solutions of PLURONIC P-105) is transferred onto the polystyrene surface, the remainder being retained within the PLURONIC micelles.

### EXAMPLE 3

Effect of Drug Encapsulation in PLURONIC Micelles on the Intracellular Uptake by HL-60 Cells

Intracellular uptake of doxorubicin and ruboxyl was measured using a fluorescence technique wherein compounds were excited at 488 nm and technical emission spectra were recorded at 510-700 nm. Two sets of samples were studied, incubated, and sonicated. Ultrasound was generated by a Sonicor SC100 sonication bath operating at 70 kHz and 37° C. Power density was controlled by adjusting the input voltage and was measured with a hydrophone.

For the first set of samples, the cells were incubated at 37° C. with doxorubicin or ruboxyl, which were either dissolved in the RPMI medium or PBS, or the drugs were solubilized in PLURONIC P-105 solutions of various concentrations. For the second set of samples, the cells were sonicated by 70 kHz ultrasound at 37° C. to assess the effect of ultrasound on the drug uptake from molecular and micellar solutions. After being incubated/sonicated with and without the drug, the cells were centrifuged, washed twice with cold PBS, resuspended in PBS, and the fluorescence spectra of cell suspensions were recorded. The fluorescence intensity of the untreated cells was subtracted from that of the drug-treated cells. Because drug fluorescence within the cells was substantially quenched, drug uptake was quantified by lysing the cells by incubating them with 1 wt % SDS solution for 1-2 hours at 37° C. This process transferred the drug from cellular components to SDS micelles. Calibration experiments showed a linear dependence of fluorescence intensity on ruboxyl and doxorubicin concentration in 1 wt % SDS solutions in the concentration range of interest. Upon the completion of cell lysis, fluorescence spectra of the lysates were recorded. To quantify the concentration of lysed cells, cell lysates were filtered through 0.2 μm filters, and their optical densities were measured by protein absorbance at 280 nm (OD 280 nm). Calibration experiments showed a linear dependence of OD 280 nm on the concentration of lysed cells. The fluorescence intensity of lysates ws normalized by OD 280 nm.

Drug sequestration in PLURONIC P-105 micelles caused a substantial decrease in drug uptake by HL-60 cells (FIG. 4). These data are representative of numerous experiments on the uptake of ruboxyl and doxorubicin from PLURONIC P-105 micelles. The uptake of the drug was somewhat enhanced at a PLURONIC concentration of 0.1%, which is below the CMC for the formation of dense micelles, indicating that PLURONIC molecules in a unimeric form or in loose aggregates enhanced the permeability of cell membranes toward the drug. Drug uptake from dense PLURONIC micelles was substantially lower than that of a free drug, indicating that dense micelles inhibited drug interaction with the cells.

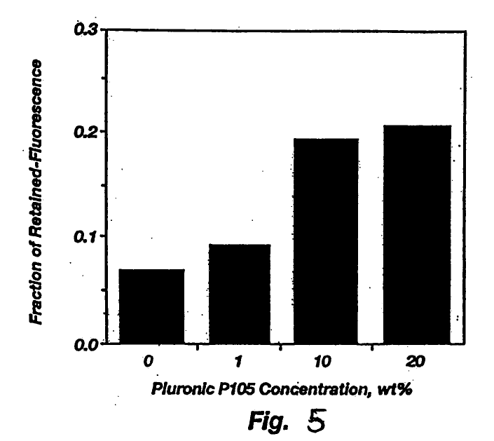

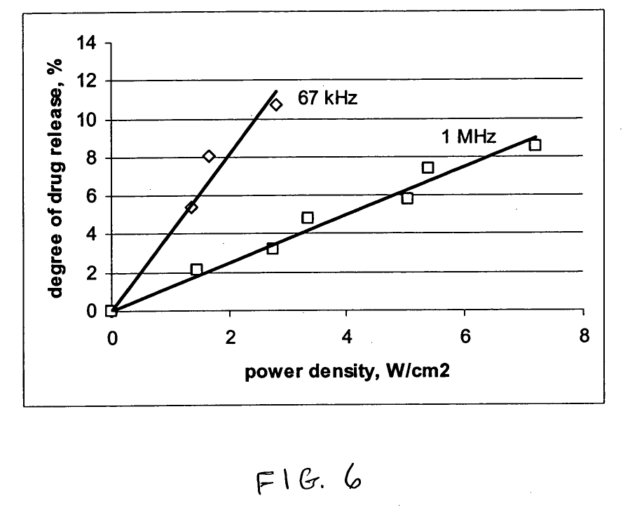

Ruboxyl and doxorubicin encapsulation in PLURONIC micelles restricted not only drug interaction with the cells, but also drug interaction with cell components, e.g. DNA. The drop of fluorescence was lower when the drug was introduced from a micellar solution, indicating a lower drug-DNA interaction (FIG. 5).

### EXAMPLE 4

This example shows the dependence of the degree of drug release from micelles on ultrasound intensity at 20 kHz and 1 MHz.

The measurements of the degree of drug release are based on the decrease of DOX fluorescence intensity when DOX is transferred from the hydrophobic environment of micelle cores to the aqueous environment. An argon-ion laser beam of 488 nm was directed to a drug-containing cuvette to excite fluorescence. The emissions were collected using a fiber optic collector and filtered to remove the excitation wavelength. The emissions were quantified using a photodetector, digitized with a 12-bit A/D converter, and stored in a Macintosh computer for further analysis.

Digitized fluorescence intensity data were analyzed to calculate the percent of the drug release from micelles. A fluorescence intensity of a 10 μg/ml DOX solution in PBS (I_{PBS}) was measured first; the PBS solution was then carefully sucked out of the cuvette and replaced with a 10 μg/ml DOX solution in 10% PLURONIC P-105 micelles. Fluorescence of this solution (I_{mic}) was measured, and a difference I_{mic}−I_{PBS} was assumed to correspond to a 100% drug release from micelles. Then, ultrasound was switched on, and DOX fluorescence under sonication (Iᵤₛ) was recorded; if sonication induced partial drug release from micelles into the aqueous environment, Iᵤₛ was lower than I_{mic}; the “ultrasound on”-“ultrasound off” cycles were repeated several times to check reproducibility. The length of each ultrasound exposure cycle was 1 to 2 minutes. The scatter of the data obtained in various ultrasound cycles did not exceed 20%. The degree of drug release (DDR) was calculated as follows:
DDR={[I_{mic}−Iᵤₛ]/[I_{mic}−I_{PBS}]}×100

The DOX release from micelles in a high-frequency range (1 MHz to 3 MHz) requires higher power densities than in a low-frequency range (20 kHz to 100 kHz). For instance, a 10% DOX release from PLURONIC P-105 micelles required a power density of 0.058 W/cm² at 20-kHz ultrasound, 2.8 W/cm² at 67-kHz ultrasound, and 7.2 W/cm² at 1.0-MHz ultrasound.

Thus, both low-frequency and high-frequency ultrasound proved effective in triggering drug release from micelles (FIG. 6). Low frequency ultrasound was found more effective in triggering drug release from micelles, but it does not allow sharp focusing. In contrast, high frequency ultrasound allows sharp focusing, but does not penetrate as deep in the interior of the body. A higher frequency ultrasound allows sharper focusing; however, drug release from micelles proceeds more effectively at lower frequencies. Typical penetration depth (the depth at which 50% of the supplied ultrasonic energy is absorbed) for 1-MHz ultrasound in various tissues is 5 cm for fat, 2.7 cm for muscle, 0.9 cm for tendon, and about 0.3 cm for bone; for 3-MHz ultrasound, penetration is about three-fold lower. In contrast, low-frequency ultrasound (20 to 100 kHz range) can penetrate to the depth of tens of centimeters in various tissue types. In this respect, high-frequency ultrasound is advantageous for targeted drug delivery to small superficial tumors while low-frequency ultrasound should be used for treating large and deeply located tumors. Therefore, optimal design of ultrasound treatment should depend on the tumor size and location. For instance, for the tumors 2-cm diameter or larger, application of 100-kHz (or a lower frequency) ultrasound appears quite feasible; optimal power densities could be achieved by controlling output energy. For smaller tumors that are not deep, a higher frequency ultrasound may be used since it provides for sharper focusing; power densities produced at the focal site by existing hyperthermia devices appear sufficient to cause drug release from micelles.

### EXAMPLE 5

Effect of Ultrasound on Intracellular Drug Uptake

A decreased uptake of the drug solubilized in dense polymeric micelles requires a method to enhance drug intracellular uptake at the tumor site. Ultrasonication of the cells in the presence of micelle-encapsulated drugs can substantially enhance intracellular uptake of the drug. Typical results on drug accumulation within the cells are presented in FIG. 7. A similar effect is observed when drug uptake is measured by depletion from the incubation medium (data not shown). The investigation of doxorubicin cytotoxicity on HL-60 cells when the drug was delivered from molecular solutions (without PLURONIC) and from micellar solutions, with and without acoustic activation, has shown that the combination of micellar delivery and ultrasonication resulted in a substantial decrease of the effective drug dose (N. Munshi et al., 117 Cancer Letters 1-7 (1997). It is noteworthy that despite a decreased intracellular uptake of the micelle-encapsulated drug, its cytotoxicity was higher than that of a free drug, probably due to the cytotoxic effect of PLURONIC micelles on mitotic cells.

### EXAMPLE 6

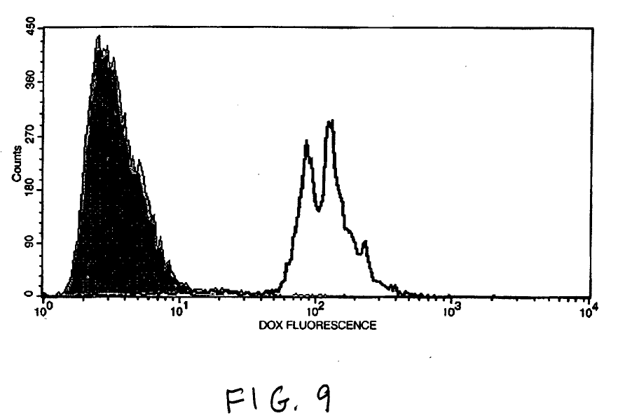

FIG. 8 shows that sonication at 1 MHz substantially increased the intracellular uptake of DOX from PBS (or RPMI 1640) by ovarian carcinoma drug-sensitive A2780 cells. Fluorescence histograms of the A2780 cells incubated or sonicated in the presence of DOX are shown. The initial concentration of cells ranged from 3×10⁶ to 5×10⁶ cells/ml as determined using a hemacytometer. After exposure to DOX (10 μg/ml to 50 μg/ml in various experiments) and ultrasound (15 to 30 s), cells were counted again to measure the degree of sonolysis, upon which they were centrifuged, washed with PBS, fixed with a 3% formalin or 2.5% glutaraldehide, and analyzed by flow cytometry. Fluorescence histograms were recorded with a FACScan flow cytometer (Beckton Dickinson) and analyzed using CellQuest software supplied by the manufacturer. A minimum of 10,000 events was analyzed to generate each histogram.

The experiments on the effect of ultrasound on the intracellular DOX uptake with and without micelles were always conducted in parallel, at the same day and using the same batch of the cells.

It should be noted that sonication in PBS caused substantial cell lysis; at a power density of 15.2 W/cm², 25% cells were lysed in PBS, whereas no cell lysis was observed in the presence of PLURONIC micelles. Cell lysis is caused by transient cavitation; sonoprotection property of PLURONIC micelles presumably results from quenching transient cavitation.

It should be noted that in these experiments, cell death was caused exclusively by ultrasound treatment rather than by the cytotoxic action of the internalized drug; at very short incubation/sonication times used in this study, drug did not affect cell viability even at much higher intracellular concentrations. However, the presence of DOX in the non-micellar systems enhanced cell lysis due to the amplified cavitation.

Note that DOX uptake by A2780 cells sonicated for only 15 s was significantly higher than that by unsonicated cells incubated in suspension with the same concentration of DOX for 30 min (FIG. 8).

### EXAMPLE 7

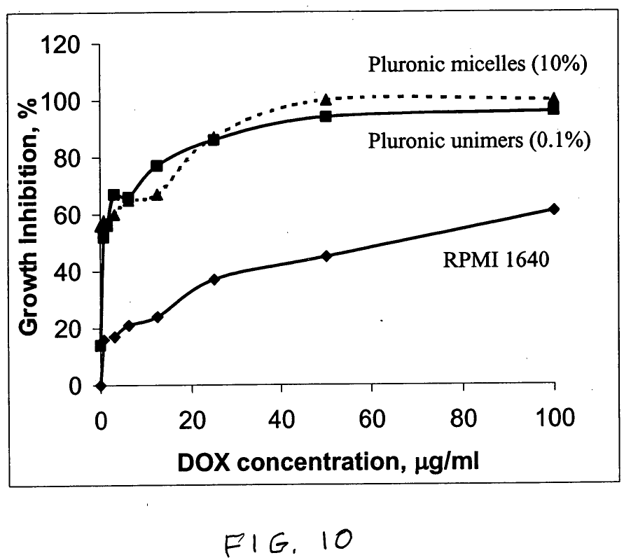

High frequency sonication enhanced the intracellular drug uptake not only by drug-sensitive but also by the multidrug resistant (MDR) cells. The enhanced uptake from both the conventional mediums (PBS or RPMI 1640) and from (or with) PLURONIC micelles was observed. Flow cytometry histograms of the MDR A2780/ADR cells unsonicated or sonicated in the presence of PLURONIC micelles are shown in FIG. 9. These data imply that micelle/ultrasound technique can provide for decreasing systemic concentration of free drug without compromising the intracellular drug uptake at the tumor site.

The enhancement of drug uptake from (or with) PLURONIC micelles under the action of 1-MHz ultrasound was observed for all cell lines studied, namely ovarian carcinoma A2780 and A2780/ADR cells, breast cancer MCF-7 cells, and leukemia HL-60 cells.

Two possible mechanisms of the ultrasound-enhanced drug uptake were proposed; one mechanism is related to the drug release from micelles while the other is associated with the enhanced uptake of the micellar-encapsulated drug. As suggested by the effect of the ultrasound pulse duration on the drug uptake, at low-frequency ultrasound, both mechanisms presumably worked in concert.

The data presented above suggest that both low-frequency and high-frequency ultrasound can effectively deliver drugs encapsulated in polymeric micelles to cancerous cells. This is an important finding since high-frequency ultrasound is widely used in clinical practice for imaging purposes (though at much lower power densities than used here). The ideal scenario for the clinical application of the above technique would be combining imaging and therapeutic ultrasound transducer arrays in one instrument that will be used first for tumor imaging followed by the automatic focusing of the therapeutic ultrasound beam.

### EXAMPLE 8

Ultrasound-induced increased intracellular uptake was accompanied by a dramatic sensitization of the MDR cells to the action of DOX (FIG. 10). Note that in a conventional medium, about 55% of the MDR cells was highly resistant to the action of DOX even at a concentration of 50 μg/ml (FIG. 10); the fraction of highly resistant cells decreased to about 30% upon a 10-min sonication by a 67-kHz ultrasound; the highly resistant fraction of the cells was completely eliminated in the presence of PLURONIC micelles; 100% MDR cells were killed by a combination of PLURONIC micelle and ultrasound at a DOX concentration as low as 0.78 μg/ml. Even without ultrasound, IC₅₀ of DOX in the MDR ovarian carcinoma cells dropped 50-fold in the presence of PLURONIC unimers and micelles.

It is important to note that the effects of PLURONIC micelles and ultrasound on drug uptake and cytotoxicity described above for DOX were also observed for another anthracycline drug, Ruboxyl. Experiments with taxol also gave promising results. This implies that the new modality of drug delivery described here will have general applicability to a wide variety of drugs and drug delivery systems.

### EXAMPLE 9

In vivo testing of the micelle/ultrasound technique using ovarian carcinoma model in nu/nu mice confirmed in vitro results. Upon A2780 cell inoculation, two internal tumors grew in untreated mice. Drug (DOX) was injected intravenously at 6 mg/kg through the tail vein. Four, eight or twelve hours upon drug injection, one of the two tumors was sonicated by 1 MHz or 3 MHz ultrasound at the output power density of 1.7 W/cm2. Ten minutes after sonication, the mouse was uthenized, tumors and other organs were excised, cut to small pieces in trypsin to produce individual cells, fixed by 2.5% glutaraldehide and evaluated by flow cytometry. Fluorescence histograms of unsonicated and sonicated A2780 tumor are presented in FIG. 11. A dramatically enhanced DOX uptake by the cells of the sonicated tumor was observed.

When 3-MHz ultrasound was used, DOX uptake by other organs was not affected by ultrasound, which dramatically enhanced tumor-to-organ DOX ratio.

### EXAMPLE 10

Several micellar delivery systems, namely PLURONIC P-105 (5%), PEG-PE (5%), and mixed micelles of 5% PLURONIC P-105 and 5% PEG-PE are compared in FIG. 12 in their ultrasound-enhanced drug delivery efficiency. Mixed micelles (bold solid line) manifested significantly higher efficiency compared to the individual components of the mixture.

### EXAMPLE 11

In accordance with higher intracellular drug uptake by tumor cells, life span of ovarian carcinoma tumor-bearing mice treated by micelle/ultrasound technique at DOX dose of 3 mg/kg was much longer than that of mice treated by conventional drug introduction (FIG. 13). A2780 cells (1×10⁶) were inoculated intraperitoneally. DOX in PBS or PLURONIC P-105 micelles was injected intraperitoneally the next day after cell inoculation. Sonication of the abdominal region was performed one hour after DOX injection.

Therefore, drug delivery in polymeric micelles combined with localized sonication of the tumor provides for drug targeting to tumors and significantly enhances the efficacy of chemotherapeutic cancer treatment. The combination of micellar drug delivery carriers and ultrasound is especially promising for treating multidrug resistant (MDR) tumors that do not react to drugs under conventional treatment regimens.

Ultrasonication enhances drug uptake from PLURONIC micelles. Based on this finding, a new concept of a localized drug delivery may be developed, based on encapsulating a drug in stabilized micelles and focusing ultrasound on the tumor.

There are two possible mechanisms of acoustically-enhanced intracellular uptake of the drug from micellar solutions: (1) acoustically-enhanced drug release from micelles and (2) acoustic effect on the permeability of cell membranes. The experiments showed that both mechanisms worked in concert.

1. A method for delivery of a hydrophobic drug to a selected site in a patient comprising:
(a) administering to said patient a composition comprising a micellar drug carrier comprising a hydrophobic core and an effective amount of said hydrophobic drug disposed in said hydrophobic core, wherein said micellar drug carrier is a member selected from the group consisting of AB-diblock copolymers, ABA-triblock copolymers, mixtures of AB-diblock copolymers and ABA-triblock copolymers, and mixtures of PEGylated diacylphospholipids and AB-diblock copolymers, ABA-triblock copolymers, or mixtures of AB-diblock and ABA-triblock copolymers; and
(b) applying ultrasound at a frequency of 20-100 kilohertz to said selected site such that said hydrophobic drug is released from said hydrophobic core to said selected site.

2. The method of claim 1 wherein the micellar drug carrier is poly(L-amino acid)-co-poly(ethylene oxide) diblock copolymer.

3. The method of claim 1 wherein the micellar drug carrier is poly(ethylene oxide)-poly(propylene oxide)-poly(ethylene oxide) triblock copolymer.

4. The method of claim 1 wherein the micellar drug carrier is a mixture of an AB-diblock copolymer and poly(ethylene oxide)-poly(propylene oxide)-poly(ethylene oxide) triblock copolymer.

5. The method of claim 1 wherein the micellar drug carrier is a mixture of a PEGylated diacylphospholipid and an AB-diblock copolymer or an ABA-triblock copolymer.

6. The method of claim 5 wherein the micellar drug carrier is a mixture of a PEGylated diacylphospholipid and poly(L-amino acid)-co-poly(ethylene oxide) diblock copolymer.

7. The method of claim 5 wherein the micellar drug carrier is a mixture of a PEGylated diacylphospholipid and poly(ethylene oxide)-poly(propylene oxide)-poly(ethylene oxide) triblock copolymer.

8. The method of claim 5 wherein the micellar drug carrier is a mixture of a PEGylated diacylphospholipid, poly(ethylene oxide)-poly(propylene oxide)-poly(ethylene oxide) triblock copolymer, and an AB-diblock copolymer.

9. The method of claim 8 wherein the PEGylated diacylphospholipid comprises 1,2-diacyl-sn-glycero-3-phosphoethanolamine-N-[methoxy(polyethylene glycol)].

10. The method of claim 5 wherein the hydrophobic drug is an anthracycline.

11. The method of claim 10 wherein the anthracycline is doxorubicin.

12. The method of claim 10 wherein the anthracycline is ruboxyl.

13. A composition comprising a micellar drug carrier comprising a hydrophobic core and an effective amount of said hydrophobic drug disposed in said hydrophobic core, wherein said micellar drug carrier further comprises a mixture of a PEGylated diacylphospholipid and an AB-diblock copolymer, an ABA-triblock copolymer, or a mixture of an AB-diblock copolymer and an ABA-triblock copolymer.

14. The composition of claim 13 wherein the PEGylated diacylphospholipid comprises 1,2-diacyl-sn-glycero-3-phosphoethanolamine-N-[methoxy(polyethylene glycol)].

15. The composition of claim 14 wherein said micellar drug carrier comprises poly(ethylene oxide)-poly(propylene oxide)-poly(ethylene oxide) triblock copolymer.

16. A method for delivery of a drug to a selected site in a patient comprising:
(a) administering to said patient a composition comprising a micellar drug carrier comprising a hydrophobic core and an effective amount of said drug disposed in said hydrophobic core, wherein said micellar drug carrier further comprises a mixture of an AB-diblock copolymer and an ABA-triblock copolymer; and
(b) applying ultrasound to said selected site such that said drug is released from said hydrophobic core to said selected site.

17. A method for delivery of a drug to a selected site in a patient comprising:
(a) administering to said patient a composition comprising a micellar drug carrier comprising a hydrophobic core and an effective amount of said drug disposed in said hydrophobic core, wherein said micellar drug carrier further comprises a mixture of a PEGylated diacylphospholipid and an AB-diblock copolymer, an ABA-triblock copolymer, or a mixture of an AB-diblock copolymer and an ABA-triblock copolymer; and
(b) applying ultrasound to said selected site such that said drug is released from said hydrophobic core to said selected site.

18. A method for delivery of a hydrophobic drug to a selected site in a patient comprising:
(a) administering to said patient a composition comprising a micellar drug carrier comprising a hydrophobic core and an effective amount of said hydrophobic drug disposed in said hydrophobic core, wherein said micellar drug carrier further comprises a mixture of 1,2-diacyl-sn-glycero-3-phosphoethanolamine-N-[methoxy(polyethylene glycol)] and poly(ethylene oxide)-poly(propylene oxide)-poly(ethylene oxide) triblock copolymer; and
(b) applying ultrasound at a frequency of 20-100 kilohertz to said selected site such that said hydrophobic drug is released from said hydrophobic core to said selected site.

19. A method of treating a multidrug resistant cancerous tumor in a patient in need thereof comprising:
(a) administering to said patient a composition comprising a micellar drug carrier comprising a hydrophobic core and an effective amount of an anticancer drug disposed in said hydrophobic core, wherein said micellar drug carrier is a member selected from the group consisting of AB-diblock copolymers, ABA-triblock copolymers, mixtures of AB-diblock copolymers and ABA-triblock copolymers, and mixtures of PEGylated diacylphospholipids and AB-diblock copolymers, ABA-triblock copolymers, or mixtures of AB-diblock and ABA-triblock copolymers; and
(b) applying ultrasound at a frequency of 20-100 kilohertz targeted to said tumor such that said anticancer drug is released from said hydrophobic core to said tumor.

20. The method of claim 19 wherein said anticancer drug comprises doxorubicin.
